(19) United States
(12) Patent Application Publication
(10) Pub. No.: US 2010/0003220 A1
(43) Pub. Date: Jan. 7, 2010

(54) AGENTS FOR TREATING MALIGNANT MESOTHELIOMA

(76) Inventors: Katsuhito Takahashi, Kyoto-shi (JP); Hisako Yamamura, Ikoma-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/309,675

(22) PCT Filed: Jul. 27, 2007
(86) PCT No.: PCT/JP2007/064798

§ 371 Date: Jan. 27, 2009

(30) Foreign Application Priority Data
Jul. 27, 2006 (JP) 2006-205006

(51) Int. Cl. A61K 45/00; C12N 5/08; C12N 7/00; A61P 35/00

(52) U.S. Cl. 424/93.6; 435/366; 435/235.1

(57) ABSTRACT

The present invention provides an effective therapeutic composition for malignant mesothelioma. Specifically, the present invention provides a therapeutic composition for mesothelioma, comprising a calponin-targeting and tumor-lysing variant of herpes simplex virus (HSV-1), preferably a strain d12.CALPfΔRR. The present invention further provides a method for generating a cell for treating mesothelioma, which comprises infecting mesothelioma cells removed from a patient with an F-type variant of herpes simplex virus proliferating with targeting a calponin gene, preferably a strain d12.CALPfΔRR. Also provided are a cell obtainable by the method, and a calponin-targeting and tumor-lysing variant of herpes simplex virus (HSV-1), preferably a strain d12.CALPfΔRR.

### TECHNICAL FIELD

The present invention relates to a treatment of mesothelioma and, in particular, malignant mesothelioma. Specifically, the present invention relates to uses of variants of herpes simplex virus proliferating with targeting a calponin gene, for the treatment of mesothelioma and, in particular malignant mesothelioma, and to such viral variants.

### BACKGROUND ART

Recently, the occurrence of malignant mesothelioma resulting from asbestos is ever increasing in developed countries. Also in Japan, the occurrence of malignant mesothelioma continues to increase, since peritoneal malignant mesothelioma was reported in 1973, and the number of deaths in 2004 reached 970, which is almost twice in comparison to that in 2002. It is estimated that patients with mesothelioma continue to increase until 2030, since the period from the first exposure to asbestos to the onset of mesothelioma is 11 to 54 years (a median value of 38.6 years), according to the report in 2003 by the Ministry of Health, Labour and Welfare, Japan, and the period of using asbestos in largest quantities in Japan was from 1970 to 1990. According to an estimated maximum number of deaths, it is likely that the number of persons who die from mesothelioma in a period of 2025 to 2030 in Japan will be more than 10,000 every year, which is comparable to the number of persons who die from breast cancer at present in Japan. Therefore, it is a current world-wide problem to develop therapeutic methods for malignant mesothelioma.

Although treatments for malignant mesothelioma have been attempted with multidisciplinary modalities combining surgical treatments, such as extrapleural pneumonectomy, chemotherapies with cisplatin, Alimta®, and the like, and radiotherapies, the prognosis is extremely poor, with a two-year survival rate of only 20 to 30%. The investigation by registering cancers from 1975 to 2001 in Osaka Prefecture (refer to non-patent document 1) also brings the result that men has a five-year survival rate of 5% and a median survival time of less than 6 months. Especially for sarcoma-type and biphasic (epithelium types+sarcoma types)-type mesotheliomas, which account for 40% of all the cases on the basis of a histopathological classification, treatments other than surgical resection are not effective, and thus there is an eager need to develop new therapeutic methods.

Gene therapy, a new area of treatments, is being established with the progress of technologies for introducing genes, including viral vectors. Gene therapies which have been attempted for malignant mesothelioma are divided broadly into four types. A first type of gene therapy is a gene therapy in which a herpes simplex virus thymidine kinase gene (HSV-tK), referred to as a suicide gene, and an adenovirus vector are employed, and a phase I clinical trial in 21 patients with malignant pleural mesothelioma was conducted in the United State and resulted in only two patients surviving for more than five years (refer to non-patent document 2). A second type is an immunogene therapy. A phase I clinical trial in which interleukin-2 was administered intrathoracically to six patients with pleural mesothelioma employing a small pox virus vector was conducted in the United State and did not provide any therapeutic effect (refer to non-patent document 3). A third type is a method by which immune responses are also induced against mesothelioma cells by intrathoracically administering non-proliferative ovarian cancer cells into which an HSV-tK gene has been gene introduced employing a retrovirus, followed by killing of the ovarian cancer cells with ganciclovir, and no therapeutic effect has been reported (refer to non-patent document 4). A fourth type is a method by which a proliferative attenuated HSV-1 from which a gene or genes responsible for pathogenesis, such as γ34.5, have been removed is used, which is still under basic experiments and has no selectivity to mesothelioma cells (refer to non-patent document 5).

Non-patent document 1: Kanazawa N., et al., Jpn. J. Clin. Oncol. 36, 254-257, 2006
Non-patent document 2: Sterman, D. H. et al., Clin. Cancer Res. 11, 7444-7453, 2005
Non-patent document 3: Mukherjee, S. et al., Cancer Gene Ther. 7, 663-670, 2000
Non-patent document 4: Harrion, L. H. et al., Ann. Thorac. Surg. 70, 407-411, 2000
Non-patent document 5: Adusumilli, P. S., et al., Cancer Biol. Ther. 5, 48-53, 2006

### DISCLOSURE OF THE INVENTION

An object to be achieved by the present invention is to provide an effective therapeutic composition for mesothelioma and, in particular, malignant mesothelioma.

The present inventors have devoted themselves to efforts for solving the above-described problems, with the result that the calponin protein which is a marker of smooth muscle cells is found to be expressed also in human malignant mesothelioma cells. In particular, the present inventors have revealed that a rabbit polyclonal antibody generated against a synthetic peptide of a carboxyl terminal region of calponin does not stain reactive mesothelial cells which are of a non-tumor tissue, but selectively stains malignant mesothelioma cells located in tumor sites. Subsequently, the present inventors have found that cultured cells of human malignant mesothelioma are effectively destroyed by d12.CALPfΔRR, which is a variant of a calponin-targeting and tumor-lysing HSV-1, d12.CALPΔRR (PCT/JP02/13683, entitled “Cell-Specific Expression/Replication Vector”). Furthermore, it has been found that administration of d12.CALPfΔRR to individuals results in remarkably decreased tumor volumes in evaluating systems of experimental treatments—in which cultured cells of malignant mesothelioma, established from human surgical specimen, are implanted into the thoracic cavity and under the skin on the back of SCID mice, whereby the present inventors have arrived at the completion of the present invention.

It is known that d12.CALPΔRR destroys human sarcoma cells with targeting calponin (PCT/JP02/13683, entitled “Cell-Specific Expression/Replication Vector”). However, this patent application does not describe or suggest the findings as set forth in the present invention.

Thus, the present invention provides:

(1) A therapeutic composition for mesothelioma, comprising an F-type variant of herpes simplex virus proliferating with targeting a calponin gene;
(2) The therapeutic composition according to (1), wherein the variant is derived from a strain d12.CALPΔRR;
(3) The therapeutic composition according to (2), wherein the variant is a strain d12.CALPfΔRR;
(4) The therapeutic composition according to any one of (1) to (3), wherein the mesothelioma is malignant;
(5) A method for generating a cell for treating mesothelioma, which comprises infecting mesothelioma cells removed from a patient with an F-type variant of herpes simplex virus proliferating with targeting a calponin gene;
(6) The method according to (5), wherein the variant is derived from a strain d12.CALPΔRR;
(7) The method according to (6), wherein the variant is a strain d12.CALPfΔRR;
(8) The method according to any one of (5) to (7), wherein the mesothelioma is malignant;
(9) A cell for treating mesothelioma, which is obtainable by the method according to any one of (5) to (8);
(10) A method for treating mesothelioma, which comprises administering to a patient with mesothelioma an F-type variant of herpes simplex virus proliferating with targeting a calponin gene;
(11) The method according to (10), wherein the variant is derived from a strain d12.CALPΔRR;
(12) The method according to (11), wherein the variant is a strain d12.CALPfΔRR;
(13) The method according to any one of (10) to (12), wherein the mesothelioma is malignant;
(14) A method for treating mesothelioma, which comprises administering to a patient with mesothelioma the cell for treating mesothelioma according to (9);
(15) Use of an F-type variant of herpes simplex virus proliferating with targeting a calponin gene, for manufacturing a medicament for the treatment of mesothelioma;
(16) The use according to (15), wherein the variant is derived from a strain d12.CALPΔRR;
(17) The use according to (16), wherein the variant is a strain d12.CALPfΔRR;
(18) The use according to any one of (15) to (17), wherein the mesothelioma is malignant;
(19) Use of the cell for treating mesothelioma according to (9), for manufacturing a medicament for the treatment of mesothelioma;
(20) An F-type variant of herpes simplex virus proliferating with targeting a calponin gene;
(21) The variant according to (20), which is derived from a strain d12.CALPΔRR;
(22) The variant according to (21), which is a strain d12.CALPfΔRR.

According to the present invention, a therapeutic composition for mesothelioma is provided, especially for a sarcoma type of malignant mesothelioma, which is hitherto believed to be utterly cureless.

### BRIEF DESCRIPTION OF THE DRAWINGS

FIG. 1 represents pictures showing immunohistochemistry with a polyclonal antibody directed to calponin, of surgical specimens of human malignant mesothelioma displaying the expression of the calponin protein selective in malignant mesothelioma (brown-stained regions). A represents reactive mesothelium, B represents a sarcoma-type malignant mesothelioma, Case 1, and C represents another sarcoma-type malignant mesothelioma, Case 2. Case 1 has a higher expression of calponin than that in normal vascular smooth muscle cells. Case 2 has a uniform expression of calponin in tumor cells. The antibody is a rabbit antiserum generated against the carboxyl terminal 18-mer peptide of human h1 calponin, Leu281-Gly-Asp-Pro-Ala-Ala-His-Asp-His-His-Ala-His-Asn-Tyr-Tyr-Asn-Ser-Ala297, and purified using a Protein A-Sepharose column (Amersham Biosciences).

FIG. 2 represents pictures after 42 hours elapsed since Vero cells were treated with a suspension of cells infected with an HSV-1 variant, d12.CALPfΔRR. The circled region in Panel (A), which is prior to separation, shows single clones of variants characterized by cell membrane-fused, syncytium-forming plaques among d12.CALPΔRR plaques characterized by forming cell-lysing plaques. The circle in Panel (B), which is after separation, shows that the variant clones in the region have completely been aspirated into chips. Panel (C), which is from an infection experiment (1) of Vero cells after separation, represents a picture obtained by observation 42 hours after the infection of Vero cells of 6.0×10⁵ cells/well (of 6-well plates) with the whole amount of a cell suspension. Panel (D), which is from an infection experiment (2) of Vero cells after separation, represents a picture taken 24 hours after Vero cells cultured in FALCON T-150 bottles were subjected to infection with 6 μl of the cell suspension obtained from the infection experiment (1).

FIG. 3 represents pictures showing the results evaluating the number and the area per well of plaques stained with X-Gal after 48 hour infection by infecting sub-confluent monolayer cultures of malignant mesothelioma cells with d12.CALPΔRR and d12.CALPfΔRR at MOIs of 0.1 and 1.0.

FIG. 4 represents pictures showing the results of immunoblot analysis of the expression of the ICP4 protein 22 hours after infecting sub-confluent monolayer cultures of human malignant mesothelioma and human leiomyosarcoma cells with d12.CALPfΔRR at MOIs of 0.01 and 0.1.

FIG. 5 represents a graph indicating the results of viral replication analysis, showing the sensitivity to ganciclovir of d12.CALPfΔRR.

FIG. 6 represents real-time in vivo imaging views showing therapeutic effects of d12.CALPfΔRR in SCID mice into which cultured cells of human malignant mesothelioma were implanted subcutaneously on the back. Panel A shows the appearance of the implanted human malignant mesothelioma from a SCID mouse into which a viral buffer was injected solely, Panel B shows the appearance of the implanted human malignant mesothelioma from a SCID mouse into which d12.CALPfΔRR injected, and Panel C shows the appearance of the implanted human malignant mesothelioma from another SCID mouse into which d12.CALPfΔRR injected. In these pictures, 1 indicates a tumor at the side where no treatment was administered, and 2 (arrows) indicates tumors at the side where a viral buffer or d12.CALPfΔRR was injected, and the circles indicate the location of respective tumors and the site where background chemiluminescence was detected. The viral buffer or d12.CALPfΔRR was injected directly into tumors three times at an interval of five days. The cultured cells of malignant mesothelioma established from human surgical specimens were labeled beforehand with a luciferase gene.

FIG. 7 represents a picture showing the expression of the LacZ gene (blue color), indicating the proliferation of d12.CALPfΔRR in human malignant mesothelioma cells implanted subcutaneously on the back of SCID mice, and the reduction in the diameter of tumors, indicating remarkable antitumor effects. The two lefts represent tumors into which a viral buffer was injected solely and the two rights represent tumors into which d12.CALPfΔRR was injected.

FIG. 8 represents pictures showing antitumor effects of d12.CALPfΔRR on human malignant mesothelioma cells implanted intrathoracically in SCID mice. Panel A shows the appearance of the intrathoracically implanted human malignant mesothelioma cells from a mouse into which a viral buffer was injected solely and Panel B shows the appearance of the intrathoracically implanted human malignant mesothelioma from a mouse into which d12.CALPfΔRR was injected. The viral buffer or d12.CALPfΔRR was injected once directly into the thoracic cavity. Remarkable antitumor effects were observed by injection of d12.CALPfΔRR.

FIG. 9 represents in vivo imaging views of luciferase labeled tumors, showing antitumor effects of d12.CALPfΔRR in a SCID-mouse intrathoracic orthotopic transplant model of human pleural malignant mesothelioma. The upper pictures represent the results of control mice into which a viral buffer was injected solely and the lower pictures represent the results of mice into which d12.CALPfΔRR was injected.

FIG. 10 represents a graph showing photon counts of luciferase labeled tumors in a treatment experiment with d12.CALPfΔRR in a SCID-mouse intrathoracic orthotopic transplant model of human pleural malignant mesothelioma. The arrows indicate the time of virus injection.

### BEST MODE FOR CARRYING OUT THE INVENTION

The present invention, in an aspect, is directed to a therapeutic composition for mesothelioma, comprising an F-type variant of herpes simplex virus proliferating with targeting a calponin gene. Variants of herpes simplex virus for use in the present invention may be variants of HSV-1 or HSV-2, if they proliferate with targeting a calponin gene, with variants of HSV-1 being preferred. In addition, variants of herpes simplex virus for use in the present invention are those which acquire the capability of fusing cells (referred to herein as “F-type” variants). F-type variants are characterized in that their infected cells form syncytia. Syncytium formation is believed to result from entering of viruses into cells, which in turn fuse with neighboring non-infected cells by the action of viral membrane proteins expressed on the cell-membrane surface of the infected cells (cell fusion requiring infection), and cytopathic effects caused by virus infection are stronger when cell-fusing plaques are formed than when cytolytic plaques are formed. F-type variants of the present invention act specifically on and results in efficient destruction of cultured cells of human mesothelioma and, in particular, human malignant mesothelioma cells. Furthermore, F-type variants of the present invention can proliferate with targeting a calponin gene, and therefore are capable of efficient destruction of tumors expressing a calponin gene, such as not only mesothelioma, but also leiomyosarcoma.

F-type variants which are employed in the present invention may be variants which are derived spontaneously from or obtained by gene modifications from herpes simplex viruses proliferating with targeting a calponin gene. Processes known for gene manipulation can be employed, in order to obtain herpes simplex viruses proliferating with targeting a calponin gene. Examples of such processes are described hereinafter. Processes known for gene manipulation can be also employed, in order to obtain F-type variants from herpes simplex viruses. Examples of such processes include, for example, those by which mutations are made in a gene selected from gB, gK, gL, UL20, and UL24 genes within the HSV-1 gene locus.

F-type variants which are preferably employed in the present invention are variants derived from the virus disclosed in PCT/JP02/13683, d12.CALPΔRR, which variants may be generated by natural mutations or obtained by gene modifications (for example, by making mutations in a gene selected from gB, gK, gL, UL20, and UL24 genes within the HSV-1 gene locus) through known processes. The parent virus, d12.CALPΔRR, is characterized by forming cytolytic plaques and F-type variants of d12.CALPΔRR are characterized in that their infected cells form syncytia (as described above). F-type variants of herpes simplex virus which are particularly preferably employed in the present invention are those which are generated by natural mutations from d12.CALPΔRR. One of the variants which are generated by natural mutations from d12.CALPΔRR is herein referred to as a “strain d12.CALPfΔRR.” In the specification, an “F-type variant of herpes simplex virus proliferating with targeting a calponin gene” may be generically termed an “F-type variant” or “virus of the present invention.”

The strain d12.CALPfΔRR was obtained on Sep. 12, 2005, and has been kept and maintained in the present inventors' laboratory.

Without intending to be bound to a particular theory, it is thought that the above-described variant viruses of the present invention as a therapeutic composition for malignant mesothelioma replicate and proliferate in specific cells expressing calponin, such as malignant mesothelioma, while specifically expressing viral genes therein, and consequently destroy the cells from the inside. Their mechanisms of destruction of tumor cells are thought to be due to 1) direct action of cell lysis and fusion by the proliferation of the viruses, 2) apoptosis of virus-infected cells, 3) induction of antitumor immunity by cytotoxic T-lymphocytes within individual bodies, and others. The variant viruses of the present invention do not injure normal cells and possess especially an endogenous thymidine kinase gene, and therefore can suppress viral proliferation at a desired time after the tumor treatment with the variant virus.

Specific embodiments, such as targeting of mesothelioma cells, of using the viruses of the present invention are described. For example, it is possible that a 444-bp transcription enhancer of the human 4F2 heavy chain (Mol. Cell Biol. 9, 2588-2597, 1989) is ligated upstream of the transcription initiation regulating region of the human calponin gene expressed specifically in smooth muscle cells and malignant mesothelioma cells (333 bp of −260 to +73, with the translation start site designated as +1) (Yamamura H. et al., Cancer Res. 61, 3969-3977, 2001), followed by ligation upstream of the ICP4 (α4) gene coding a transcription factor necessary for the initiation of viral replication, downstream of which foreign genes, such as an Enhanced Green Fluorescent Protein gene (U.S. Pat. No. 5,625,048) and others, are ligated via an Internal Ribosomal Entry Site (IRES) (U.S. Pat. No. 4,937,190). Homologous recombination with a gene locus necessary for viral DNA replication, preferably with the ribonucleotide reductase gene locus (ICP6, UL36), can lead to selective expression of ICP4 in specific proliferating cells, such as malignant mesothelioma cells which actively proliferate while expressing a calponin gene, and induction of viral proliferation. For monitoring of viral proliferation, marker genes, such as LacZ, may be inserted into the virus of the present invention. For example, a LacZ labeling gene may be inserted upstream of the 4F2 heavy chain transcription enhancer in the homologous recombination with ICP6. The expression of the LacZ gene is regulated by the endogenous promoter of the ICP6 gene (see, the gene construction of the virus d12.CALPΔRR disclosed in PCT/JP02/13683).

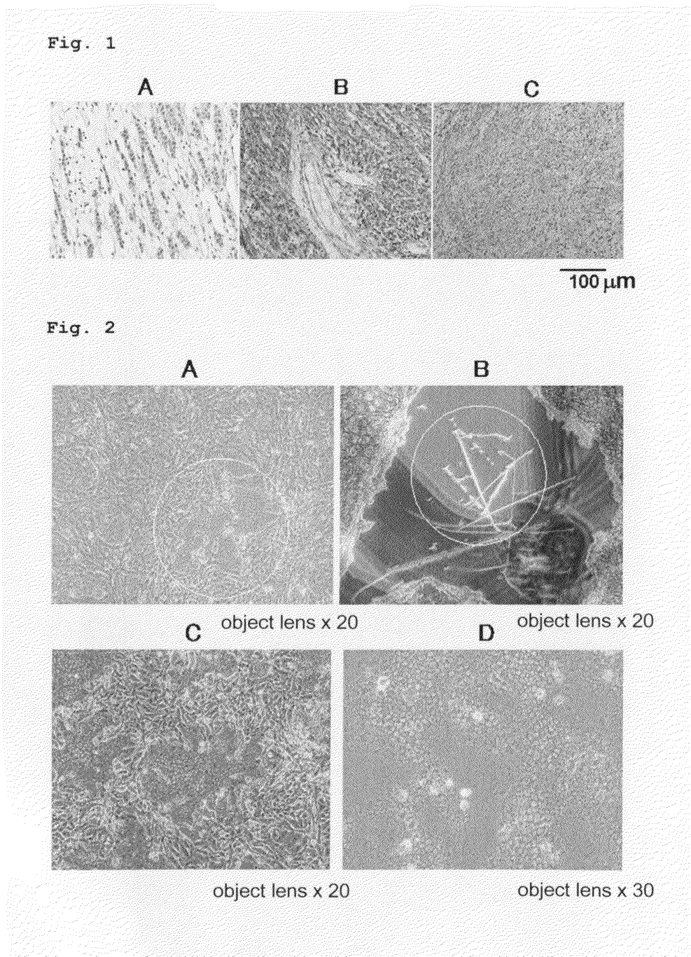

Promoters for the human calponin gene which are used in the present invention are preferably ones which regulate the expression of genes coding calponin 1 (h1 or basic calponin proteins (hereinafter referred to as calponin). Calponin was found as a troponin-like actin-binding protein present mainly in mammalian smooth muscle cells (Takahashi K. et al., Hypertension 11, 620-626, 1998), and its expression is specific in smooth muscle cells and various sarcoma cells in adults (Takahashi K. & Yamamura H., Adv. Biophys. 37, 91-111, 2003). However, the inventors have recently found that the expression of calponin and also of SM22, a troponin-like protein specific for smooth muscle cells, is observed in surgical specimens of human malignant mesothelioma and in cultured cells of malignant mesothelioma established from surgical specimens. Especially, the expression of the calponin gene have been identified in most of the cells of the sarcoma type of malignant mesothelioma, not in both normal and reactive mesothelial cells, and thus is considered to be a superior marker for targeting malignant mesothelioma cells and, in particular, the sarcoma type of malignant mesothelioma for which effective therapeutic approaches are not available at present. In the present invention, therefore, it is possible to use promoter sequences of genes coding above-described human calponin or calponin-like proteins (for example, SM22) (Yamamura H., J. Biochem. 122, 157-167, 1997). FIG. 1 shows the expression of the calponin protein in tumor cells from patients with sarcoma-type malignant mesothelioma.

In addition, promoters targeting mesothelioma cells which can be employed in the present invention are not limited to promoters of the calponin or SM22 gene as described above, and may be promoters from the group of genes whose expression is elevated mainly in epithelium-type malignant mesothelioma, relative to normal mesothelial cells, as reported by Singhal S. et al. (Singhal, S. et al., Clin. Cancer Res. 9, 3080-3097, 2003, Table 2) (94% of the cases by Singhal et al. are of an epithelium type or biphasic (epithelium and sarcoma types), which promoters allow for targeting the epithelium type of malignant mesothelioma. It is particularly preferable that the expression of such genes in normal cells is restricted to non-proliferated cells, as the calponin gene is expressed in non-proliferated normal smooth muscle cells, with the exception of mesothelioma and leiomyosarcoma cells. Table 1 provides a list of the group of genes whose expression is elevated in malignant mesothelioma cells, cited from the report by Singhal et al.

TABLE 1

| | | | |
|---|---|---|---|
| GenBank accession no. | Gene name | Fold change | P |

Cytoskeletal reorganization

| | | | |
|---|---|---|---|
| H15446 | Annexin VII (synexin) | 12.10 | 0.00349 |
| AA668178 | Karyopherin α3 | 11.69 | 0.00092 |
| AA235002 | Annexin VIII | 11.68 | 0.00759 |
| AA485668 | Integrin β4 | 10.58 | 0.00805 |
| AA598517 | Keratin 8 | 9.35 | 0.06881 |
| AA664179 | Keratin 18 | 8.90 | 0.05876 |
| N67487 | Microfibrillar-associated protein 2 | 8.60 | 0.00667 |
| H90899 | Desmoplakin I & II | 7.95 | 0.00134 |
| T98612 | Collagen III, α1 | 6.08 | 0.00410 |
| AA425450 | Neuromedin B (Integrin αE) | 5.84 | 0.00394 |
| AA451895 | Annexin V (endonexin II) | 5.75 | 0.00241 |
| AA219045 | Microtubular associated protein 1b | 5.75 | 0.00957 |
| AA419015 | Annexin IV | 5.72 | 0.00624 |
| AA490172 | Collagen I, α2 | 5.55 | 0.00481 |
| AA160507 | Keratin 5 | 5.32 | 0.02919 |
| AA487427 | Rho GDP dissociation inhibitor | 5.18 | 0.01831 |
| AA464982 | Annexin XI | 5.02 | 0.00537 |
| AA463257 | Integrin α2 | 4.78 | 0.00194 |
| H99676 | Collagen IV, α1 | 4.48 | 0.00311 |
| R40850 | α-centractin | 4.42 | 0.00047 |
| T60117 | α-spectrin | 4.25 | 0.00177 |
| AA888148 | Tubulin β2 | 3.97 | 0.00155 |
| AA634103 | Thymosin β4 | 3.69 | 0.00028 |
| AA677534 | Laminin | 3.62 | 0.00326 |
| AA634006 | α2 actin | 3.43 | 0.00304 |
| R76314 | rho G | 3.40 | 0.00316 |
| AA188179 | Arp2/3 protein complex subunit p41-Arc (ARC41) | 2.66 | 0.00013 |
| AA485959 | Keratin 7 | 2.66 | 0.04224 |
| AA464748 | Collagen VI, α2 | 2.58 | 0.01141 |
| H94892 | ral-A | 2.36 | 0.00015 |
| AA626787 | rac | 2.32 | 0.00012 |
| AA486321 | Vimentin | 2.31 | 0.01496 |
| R44290 | β-actin | 2.28 | 0.00058 |

Protein synthesis

| | | | |
|---|---|---|---|
| AA676471 | Eukaryotic translation initiation factor (eIF3) | 8.65 | 0.00175 |
| R43973 | Elongation factor 1γ | 6.02 | 0.00142 |
| H09590 | Eukaryotic initiation factor (EIF) 4AI | 5.82 | 0.00466 |
| R54097 | Translational initiation factor 2β (eIF-2β) | 4.40 | 0.00013 |
| AA669674 | Eukaryotic Translation initiation factor (EIF) 3 | 3.30 | 0.00026 |
| R43766 | Eukaryotic translation elongation factor 2 | 3.09 | 0.00087 |
| R37276 | Eukaryotic initiation factor (eIF) 4G1 | 2.43 | 0.00005 |

Metabolic pathways

| | | | |
|---|---|---|---|
| AA676466 | Argininosuccinate synthetase | 9.06 | 0.03367 |
| R15814 | Malate dehydrogenase | 7.56 | 0.00152 |
| AA664284 | Ubiquinol-cytochrome c reductase | 6.45 | 0.00013 |
| H16958 | Glyceraldehyde 3-phosphate dehydrogenase (GAPDH) | 6.41 | 0.00077 |
| H05914 | Lactate dehydrogenase-A (LDH-A) | 5.53 | 0.00001 |
| AA447774 | Cytochrome c1 | 5.40 | 0.00282 |
| N93053 | Cytochrome c oxidase | 4.99 | 0.00089 |
| R12802 | Ubiquinol-cytochrome c reductase core protein II | 4.69 | 0.00255 |
| AA455235 | Aldehyde dehydrogenase 6 | 3.98 | 0.00990 |
| AA599187 | Phosphoglycerate kinase 1 | 3.92 | 0.00110 |
| AA775241 | Aldolase A | 2.61 | 0.00082 |

Genes with potential therapeutic and prognostic implications

| | | | |
|---|---|---|---|
| AA598676 | Reticulocalbin 2 | 12.74 | 0.00041 |
| AA629897 | Laminin receptor (67 kD) | 11.56 | 0.00181 |
| AA458861 | Death associated protein | 10.94 | 0.00000 |
| AA485353 | Galectin-3 binding protein | 10.38 | 0.00026 |
| AA156461 | Pituitary tumor-transforming (PTT) interacting protein | 9.46 | 0.00004 |
| H99170 | Calreticulin (precursor) | 6.69 | 0.00162 |
| T66814 | Voltage-dependent anion channel (VDAC) 2 | 6.56 | 0.00046 |
| AA044059 | Voltage-dependent anion channel (VDAC) 1 | 5.99 | 0.00025 |
| AA158991 | Lung resistance related protein (major vault protein) | 5.54 | 0.00001 |
| AA394136 | PCTAIRE 3 | 5.42 | 0.00095 |
| W56266 | Mitogen-activated protein kinase kinase kinase 8 (MAPKKK 8) | 5.28 | 0.01120 |

A list of the group of genes whose expression is elevated mainly in epithelium-type malignant mesothelioma cells, relative to normal mesothelial cells (cited from Singhal, S. et al., Clin. Cancer Res. 9, 3080-3097, 2003)

Therapeutic compositions of the present invention for mesothelioma which comprise an F-type variant of herpes simplex virus proliferating with targeting a calponin gene are effective for every mesothelioma, such as pleural, peritoneal, and pericardial mesotheliomas. Therapeutic compositions of the present invention for mesothelioma which comprise an F-type variant are effective not only for benign mesothelioma, but also for malignant mesothelioma. The therapeutic compositions of the present invention are a breakthrough in that they are effective for treating, in particular, malignant mesothelioma.

Therapeutic compositions of the present invention for mesothelioma may take a variety of dosage forms, depending on the mode of treatments, and in general are formulated into liquid dosage forms for injection and infusion. Liquid dosage forms can be manufactured by dissolving or suspending the virus of the present invention in aqueous carriers, for example, water, saline, glucose solution, Ringer's solution, buffers, such as phosphate buffer, or the like. Therapeutic compositions of the present invention for mesothelioma may be injected directly into affected areas or administered by intravenous injection or by dripping of infusions. These methods and routes of administration can be selected as appropriate by a physician, depending on factors, such as the condition of a patient and the nature of mesothelioma (focus site, focal or diffuse, and others). The amount of virus and the number of doses to be administered can be also selected as appropriate by a physician, depending on factors, such as the condition of a patient and the nature of mesothelioma.

As a mode of administration of the present therapeutic compositions for malignant mesothelioma, the present therapeutic compositions can be administered by direct injection to primary tumor foci or to expected metastasis sites. Therapeutic compositions of the present invention can be also administered by local injection into the thoracic and peritoneal cavities, and by topical administration, such as intravascular administration to tumor feeding arteries. Alternatively, therapeutic compositions of the present invention can be also administered by intravenous injection or infusion. It is also possible to adopt modes of administration combined with needling techniques in radiofrequency ablation, catheterization techniques, surgical operations, and the like, in administrating the present therapeutic compositions for malignant mesothelioma. In addition, it can be possible that an affected site is exposed by a surgical operation and the virus of the present invention is then injected into, dripped to, or mixed into a gelatinous substrate to be contacted with the site. In therapeutic compositions of the present invention for mesothelioma, the virus of the present invention may be in a free state and supported, for example, on biocompatible or biodegradable carriers. These carriers may be ones suitable for delivery to affected sites and foci, such as the thoracic and peritoneal cavities. Such carriers can be selected as appropriate by a physician, depending on the condition of a patient, the nature of mesothelioma, and others.

In therapeutic compositions of the present invention for mesothelioma, one or more antitumor materials may be used in combination, in addition to the virus of the present invention. Antitumor materials which can be used in combination with the virus of the present invention include, but are not limited to, anticancer agents, vaccines having an action of activating innate immunity, such as BCG, antiangiogenic agents, molecular targeting drugs, radiation, heavy particle beams, and others. Herein, by “used in combination” is meant that the virus of the present invention and one or more antitumor materials may be mixed in the same therapeutic composition for mesothelioma, or that a therapeutic composition for mesothelioma comprising the virus of the present invention and a therapeutic composition or procedure comprising one or more antitumor materials may be administered separately or used in combination.

Methods and routes for administering the virus of the present invention are not limited to those described above and can be selected as appropriate by a physician.

The amount of the present therapeutic compositions for malignant mesothelioma to be administered varies with the age, sex, and condition of a patient, the disease stage, the route of administration, the number of doses, the dosage form. In general, a range of about 1×10⁶ to 1×10⁸ PFU (plaque forming units) is appropriate as a titer of HSV-1 virus per dose for adults. The present therapeutic compositions for malignant mesothelioma are believed to have low toxicity to normal cells and thus increased safety, since they target and destroy proliferating malignant mesothelioma cells expressing calponin. The present therapeutic compositions for malignant mesothelioma are also believed to be highly safe, because they possess an endogenous thymidine kinase gene and therefore the proliferation of the virus can be suppressed with commercially available acyclovir or paracyclovir after the treatment. In addition, FIAU, which is a uracil derivative labeled with ¹²⁵I, can be employed to detect and determine the thymidine kinase activity in vivo by Positron Emission Tomography (Bennet J. et al., Nature Med. 7, 859-863, 2001), which is helpful in further increasing safety.

The present invention, in another aspect, relates to a method of generating a cell for treating mesothelioma, characterized in that cells removed a patient itself or a relative thereof are infected with an F-type variant of herpes simplex virus proliferating with targeting a calponin gene, that is, a virus of the present invention. In another aspect, the present invention further relates to a cell for treating mesothelioma which is obtainable by such a method. Preferably, cells which are to be infected with the virus of the present invention are cells derived from a patient itself, and preferably cells collected from mesothelioma cells of a patient. Cells also may be cells collected from malignant mesothelioma. Among viruses of the present invention which are used for cell infection, one of preferable viruses is a strain d12.CALPfΔRR. Methods for infecting cells with viruses of the present invention can be methods known to those skilled in the art. Such methods include, for example, methods by which mesothelioma cells cultured in sterilized culture dishes and the virus of the present invention are incubated at a given ratio of the numbers of cells and virus particles, and can be selected as appropriate by a physician, depending on factor, such as the condition of a patient and the nature of mesothelioma. The cells thus infected with the virus of the present invention are within the scope of the present invention as well. By returning cells infected with the virus of the present invention back into the patient and preferably into the mesothelioma site, the mesothelioma in the patient, in particular, malignant mesothelioma, can be treated. Cells infected with the virus of the present invention can be injected directly into the affected site through the skin, or administered by intravenous injection or infusion. Alternatively, it is possible that an affected site is exposed by a surgical operation and cells infected with the virus of the present invention are contacted with the site by injection or dripping.

Other treatments for mesothelioma can be used in combination, in treating mesothelioma in a patient employing cells infected with the virus of the present invention, as described above.

In still another aspect, the present invention relates to an F-type variant of herpes simplex virus proliferating with targeting a calponin gene and, in particular, to a strain d12.CALPfΔRR. Such a variant is novel and exerts effects in treating mesothelioma and, in particular, malignant mesothelioma. Such a variant can be also employed for treating leiomyosarcoma.

In still another aspect, the present invention relates to a method for treating mesothelioma in a patient, which comprises administering to a patient with mesothelioma an F-type variant of herpes simplex virus proliferating with targeting a calponin gene. A preferable variant for use in the treating method is a strain d12.CALPfΔRR. Said treating method is particularly effective in treating malignant mesothelioma.

In addition, the present invention also provides a method for treatment of mesothelioma, which comprises administering to a patient with mesothelioma a cell for treating mesothelioma which is obtainable by the above-described methods. A preferable cell for treating mesothelioma which can be employed in the above-described treating method is a cell obtainable using a strain d12.CALPfΔRR. Said treating method is effective in treating, in particular, malignant mesothelioma.

In still another aspect, the present invention relates to a use of an F-type variant of herpes simplex virus proliferating with targeting a calponin gene, in manufacturing a medicament for the treatment of mesothelioma in a patient. A preferable variant for this use is a strain d12.CALPfΔRR. The present invention relates to use of an F-type variant of herpes simplex virus proliferating with targeting a calponin gene, preferably, of a strain d12.CALPfΔRR, in manufacturing a therapeutic composition, in particular, for malignant mesothelioma.

In addition, the present invention also relates to a use of a cell for treating mesothelioma which is obtainable by the above-described methods, in manufacturing a medicament for the treatment of mesothelioma. A preferable cell for treating mesothelioma which is employed for this use is one which is obtainable using a strain d12.CALPfΔRR. Such a use is suitable for manufacturing a medicament for treating, in particular, malignant mesothelioma

The following further describes the present invention specifically and in detail with reference to Examples, which are merely for the purpose of illustration and description and are not intended to limit the present invention thereto.

### Example 1

### A. In Vitro Analyses of Injury of Malignant Mesothelioma Cultured Cells and Viral Replication

1. Separation of a Syncytium-Forming HSV-1 Variant, d12.CALPfΔRR

Variant viruses forming syncytium-type plaques were found when Vero cells were infected with d12.CALPΔRR forming cytolytic plaques. These viruses, along with cells, were aspirated using GILSON Pipetman® P200 chips and separated. The resulting viruses were suspended in 100 μl of a cold viral buffer (20 mM Tris-HCl, pH 7.5 containing 150 mM NaCl), and frozen and stored.

FIG. 2 represents pictures from an infection experiment (1) in which Vero cells of 6.0×10⁵ cells/well (of 6-well plates) were infected with the whole quantity of 100 μl of the above-described cell suspension and observation was performed 42 hours later. A culture medium was removed from infected cells in one well of a 6-well plate. A cell suspension was prepared in 1.5 ml of a GIBCO/BRL serum-free medium VP-SFM and centrifuged for 5 minutes at 3,000 rpm, after that the supernatant was centrifuged for 45 minutes at 20,000 rpm. The precipitated fraction was suspended in 20 μl of the cold viral buffer, and frozen and stored at −80° C. FIG. 2, which also represents an infection experiment (2), presents pictures obtained 24 hours after Vero cells cultured in FALCON T-150 bottles were infected with 6 μl of the cell suspension obtained from the infection experiment (1), which allowed one to confirm that every plaque formed by the separated viruses was a syncytium-type plaque.

### 2. Analysis of Injury of Human Malignant Mesothelioma Cultured Cells

Sub-confluent monolayer cultures of malignant mesothelioma cells in 6-well tissue culture plates were infected, in 1% heat-inactivated FBS/PBS, with d12.CALPΔRR and d12.CALPfΔRR at multiplicities of infection (MOIs) of 0.1 to 1.0 pfu/cell. These infected cells were incubated at 37° C. for 1 hour and then cultured in the above-mentioned medium containing 1% FBS and 11.3 μg/ml of human IgG (Jackson ImmunoResearch Lab.). Forty-eight hours after infection, staining with X-Gal was carried out to assess the number and the area per well of plaques. The results are shown in FIG. 3. D12.CALPfΔRR displayed more potent cytotoxic activities against the cultured cells of human malignant mesothelioma, as compared to d12.CALPΔRR (blue color by X-Gal staining indicates regions of viral proliferation and cellular injury).

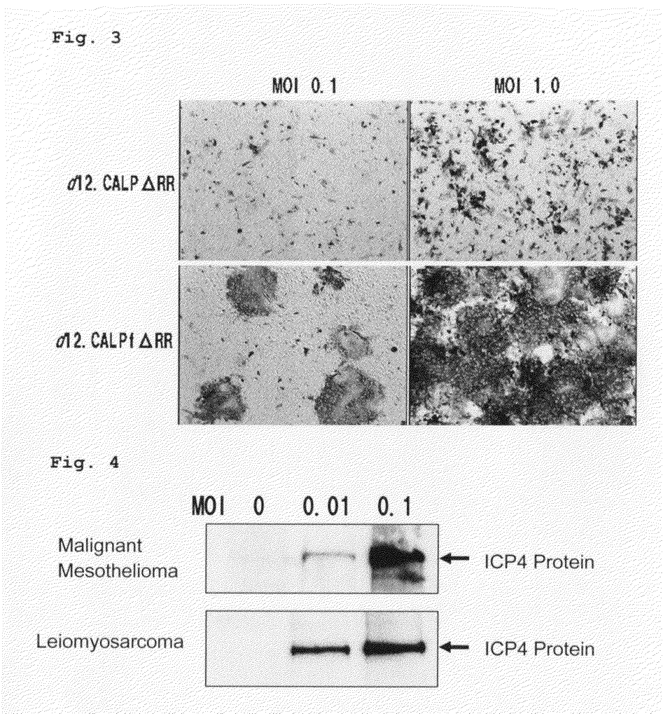

For immunoblot analysis of the expression of the ICP4 protein, respective cultured cells of human malignant mesothelioma and human leiomyosarcoma were infected with d12.CALPfΔRR at multiplicities of infection (MOIs) of 0.01 to 0.1 and cultured for 22 hours, and then cell extracts were collected. The same amount of total proteins was subjected to 9% SDS-PAGE gel electrophoresis and transferred to nitrocellulose membranes (Bio-Rad). The membranes were blocked with 5% skim milk (DIFCO Laboratories) at room temperature for 2 hours, and then incubated with an anti-ICP4 antibody (Goodwin Institute for Cancer Research; dilution of 1:500) overnight at 4° C. The results are shown in FIG. 4. The expression of the ICP4 protein, which indicates viral proliferation, was observed both in malignant mesothelioma cells and in leiomyosarcoma cells, depending on the MOIs. These results have shown that the virus variant of the present invention acts on calponin expressing tumors, such as not only mesothelioma, but also leiomyosarcoma, and can suppress these tumors.

3. Analysis of Viral Replication Showing Ganciclovir sensitivity of d12.CALPfΔRR

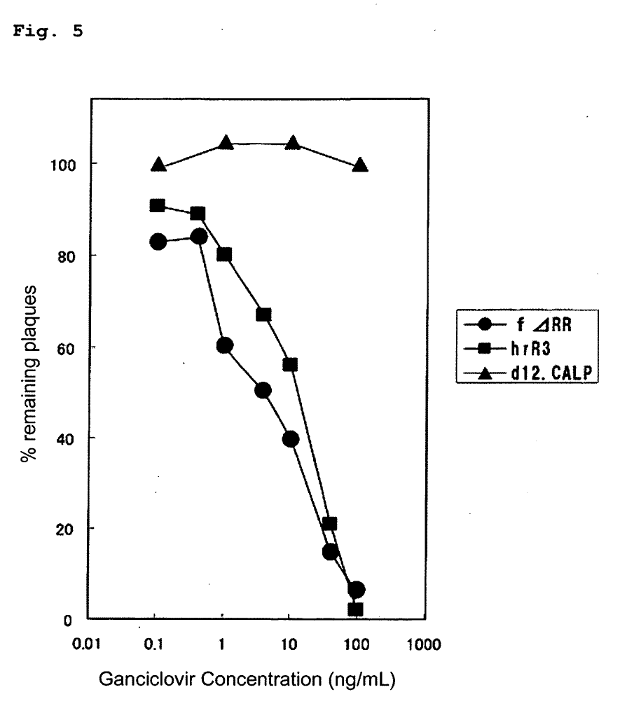

5×10⁴ cells/well of an SK-LMS-1 cultured cell line of human leiomyosarcoma were cultured in 24-well culture plates and infected with d12.CALPfΔRR, d12.CALP and hrR3 viruses at a MOI of 0.01, and then ganciclovir was added to 1% FBS/DMEM at various concentrations, followed by 26 hour culturing. Cells were fixed in 10% formalin/PBS and then stained with X-Gal to count the number of plaques. The results are shown in FIG. 5. D12.CALPfΔRR displays a higher sensitivity to ganciclovir than hrR3, an ICP6-deficient HSV-1 variant having high sensitivity to ganciclovir, and therefore is found to be highly safe. D12.CALP, an HSV-1 variant deficient in the endogenous thymidine kinase gene, possessed no sensitivity to ganciclovir

### B. In Vivo Treatments and Immunohistochemical Analysis

1. Studies on Therapeutic Effects by Three Intratumoral Administrations of d12.CALPfΔRR, on a Model in which Human Malignant Mesothelioma Cells were Subcutaneously Implanted.

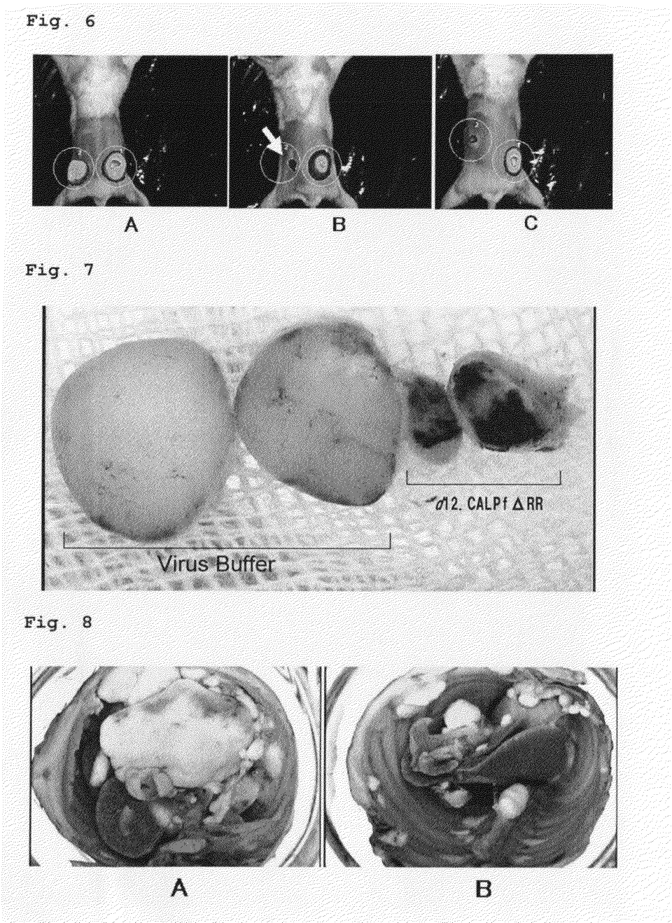

A luciferase gene was transfected into human malignant mesothelioma cells and a clone was selected which had the highest chemiluminescence intensity and the highest proliferation rate. Mesothelioma tumor blocks of measuring 4 to 5 mm per side, which were derived from a subcutaneous colony of cloned cells on the back in SCID mice, were implanted subcutaneously on the back in 6-week-old female severe combined immunodeficiency (SCID) mice (Japan SLC). The tumors grew to a diameter of 6 to 7 mm or so (50-70 mm³) in 30 days after their implantation into SCID mice. A total of 3 intratumoral injections at an interval of 5 days were administered employing 30 G needles, with 50 μl (per mouse) of a viral buffer containing 1×10⁷ pfu/mouse of d12.CALPfΔRR (n=3), or with the same volume of the viral buffer (n=3). At day 11 after the first injection of the virus, luciferin (Sigma Chemicals) was injected intraperitoneally and chemiluminescence from subcutaneous tumor cells on the back (total photon counts) was measured with a high-sensitive CCD camera using a real-time in vivo imaging system (Berthold). The results from real-time in vivo imaging are shown in FIG. 6. Control (tumors injected with the viral buffer) had a photon count of 2.79×10⁷, and the treatment group (tumors injected with d12.CALPfΔRR) had a photon count of 3.84×10⁶ and reduced the photon count to 13.7% of that of the control. These results revealed that d12.CALPfΔRR had remarkable antitumor effects by its direct injection to human malignant mesothelioma cells which had been implanted subcutaneously on the back.

For histological studies, subcutaneous tumors were removed at predetermined time points after the injection of d12.CALPfΔRR and fixed overnight at 4° C. with 2% paraformaldehyde and 0.5% glutaraldehyde, in PBS containing 1 mM MgCl₂. Subsequently, the tumors were immersed for 3 hours at 37° C. in a substrate solution containing X-gal (1 mg/ml), 5 mM K₃Fe(CN)₆, 5 mM K₄Fe(CN) 6, and 1 mM MgCl₂ in PBS, and then washed in PBS containing 3% DMSO. The results are shown in FIG. 7. The expression of the LacZ gene, which indicates viral proliferation, and remarkable antitumor effects were observed in malignant mesothelioma into which d12.CALPfΔRR was injected.

2. Studies on Therapeutic Effects by Two Intrathoracic Administrations of d12.CALPfΔRR, on a Model in which Human Malignant Mesothelioma Cells were Intrathoracically Implanted.

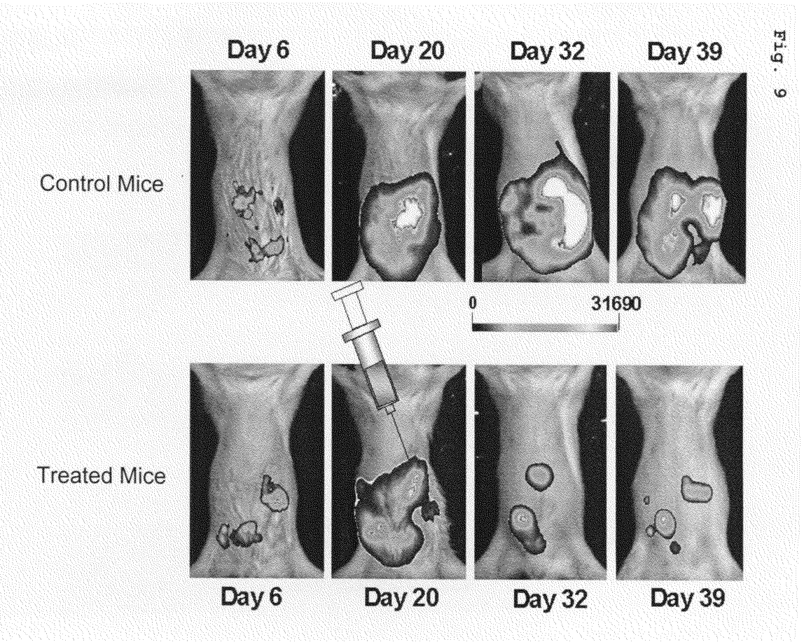

Malignant mesothelioma cells (1×10⁷ cells/mouse) were injected intrathoracically in 6-week-old female SCID mice to generate an intrathoracic malignant mesothelioma model. At 2 weeks after the implantation into SCID mice, a total of 2 intrathoracic injections at an interval of 1 week were administered employing 30-G needles, with 50 μl (per mouse) of a viral buffer containing 1×10⁷ pfu/mouse of d12.CALPfΔRR (n=3), or with the same volume of the viral buffer (n=3). At day 19 after the first injection of the virus, the mice were dissected to evaluate therapeutic effects. The results are shown in FIG. 8. D12.CALPfΔRR showed remarkable antitumor effects on human malignant mesothelioma cells which had been implanted intrathoracically in SCID mice.

3. Studies on Therapeutic Effects on an SCID-Mouse Intraperitoneal Orthotopic Transplant Model of Human Peritoneal Malignant Mesothelioma by Three Intraperitoneal Administrations of d12.CALPfΔRR

The luciferase gene pGL4.13 (Promega) derived from firefly was introduced into cultured cells of human peritoneal malignant mesothelioma, and luciferase-labeled cell line of human peritoneal malignant mesothelioma was obtained using an in vitro imaging method. This cell line was used to produce a luciferase-labeled intraperitoneal model of human peritoneal malignant mesothelioma. In vivo imaging was performed at days 6, 20, 32, and 39 after the intraperitoneal implantation of tumors. Luciferin (3.75 mg/kg) was intraperitoneally administered under diethyl ether anesthesia. Nembutal (25 mg/kg) was intraperitoneally administered 3 minutes after the intraperitoneal administration of luciferin. After 10 minutes, imaging was started using NightOWLs (Berthold). At days 21, 27, and 33 after the intraperitoneal implantation of tumors, the d12.CALPfΔRR virus was administered directly into the peritoneal cavity. The d12.CALPfΔRR virus was diluted in a viral buffer to 1.0×10⁷ pfu/100 μL and administered into the peritoneal cavity at a volume of 100 μL per mouse under diethyl ether anesthesia (n=5). For the control group, the viral buffer was administered directly into the peritoneal cavity at a volume of 100 μL per mouse (n=5).

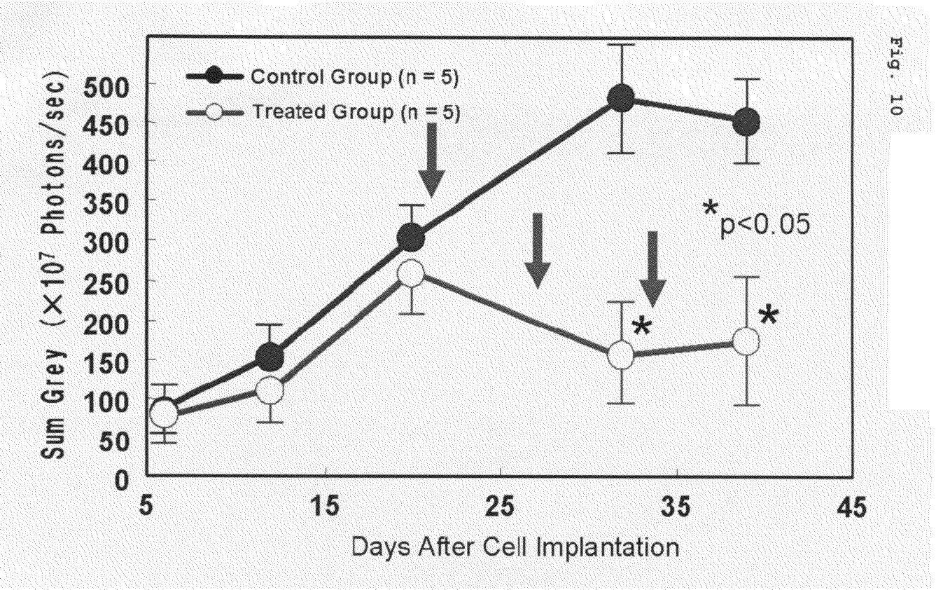

In the control group, the intensity of luminescence was increased with growing tumors, at day 11 after the first administration of the viral buffer. In the group receiving the d12.CALPfΔRR virus, on the other hand, the intensity of luminescence was decreased at days 11 and 18 after the first administration of the virus, which confirmed that there was a positive antitumor effect by the administration of the virus (FIG. 9). When comparing changes in photon counts between before and after the administration of the virus, the group receiving the d12.CALPfΔRR virus had a significant decrease in photon counts, which confirmed antitumor effects by the virus (FIG. 10). There was observed no significant differences in photon counts between the control group and the d12.CALPfΔRR virus group before the administration of the virus. The photon count immediately before removing the tumors was 1.98 times higher in the control group than in the d12.CALPfΔRR virus group. In addition, the weight of the removed intraperitoneal tumors was 2.1 times heavier in the control group than in the group receiving the d12.CALPfΔRR virus. This indicates that the photon count well reflected the intraperitoneal tumor volume in mice. These results confirmed therapeutic effects by the d12.CALPfΔRR virus in this orthotopic transplant model.

### INDUSTRIAL APPLICABILITY

The present invention can be used in fields of therapeutic drugs for malignant mesothelioma and other applications.

1. A therapeutic composition for mesothelioma, comprising an F-type variant of herpes simplex virus proliferating with targeting a calponin gene.

2. The therapeutic composition according to claim 1, wherein the variant is derived from a strain d12.CALPΔRR.

3. The therapeutic composition according to claim 2, wherein the variant is a strain d12.CALPfΔRR.

4. The therapeutic composition according to claim 1, wherein the mesothelioma is malignant.

5. A method for generating a cell for treating mesothelioma, which comprises infecting mesothelioma cells removed from a patient with an F-type variant of herpes simplex virus proliferating with targeting a calponin gene.

6. The method according to claim 5, wherein the variant is derived from a strain d12.CALPΔRR.

7. The method according to claim 6, wherein the variant is a strain d12.CALPfΔRR.

8. The method according to claim 5, wherein the mesothelioma is malignant.

9. A cell for treating mesothelioma, which is obtainable by the method according to claim 5.

10. A method for treating mesothelioma, which comprises administering to a patient with mesothelioma an F-type variant of herpes simplex virus proliferating with targeting a calponin gene.

11. The method according to claim 10, wherein the variant is derived from a strain d12.CALPΔRR.

12. The method according to claim 11, wherein the variant is a strain d12.CALPfΔRR.

13. The method according to claim 10, wherein the mesothelioma is malignant.

14. A method for treating mesothelioma, which comprises administering to a patient with mesothelioma the cell for treating mesothelioma according to claim 9.

15. Use of an F-type variant of herpes simplex virus proliferating with targeting a calponin gene, for manufacturing a medicament for the treatment of mesothelioma.

16. The use according to claim 15, wherein the variant is derived from a strain d12.CALPΔRR.

17. The use according to claim 16, wherein the variant is a strain d12.CALPfΔRR.

18. The use according to claim 15, wherein the mesothelioma is malignant.

19. Use of the cell for treating mesothelioma according to claim 9, for manufacturing a medicament for the treatment of mesothelioma.

20. An F-type variant of herpes simplex virus proliferating with targeting a calponin gene.

21. The variant according to claim 20, which is derived from a strain d12.CALPΔRR.

22. The variant according to claim 21, which is a strain d12.CALPfΔRR.
